(19) United States
(12) Patent Application Publication
(10) Pub. No.: US 2024/0065584 A1
(43) Pub. Date: Feb. 29, 2024

(54) Dual-Mode Biosensor

(71) Applicant: Meta Materials Inc., North Billerica, MA (US)

(72) Inventors: Ahmed Teyeb, Uxbridge (GB); Jamil Kanfoud, Uxbridge (GB); Tat Hean Gan, Uxbridge (GB); Andrew Mark, London (GB); Helena Cano-Garcia, London (GB); Efthymios Kallos, London (GB); Fergus O'Brien, London (GB)

(21) Appl. No.: 18/241,829

(22) Filed: Sep. 1, 2023

Related U.S. Application Data
(63) Continuation-in-part of application No. PCT/EP2022/054952, filed on Feb. 28, 2022.

(30) Foreign Application Priority Data
Mar. 1, 2021 (GB) 2102880.8
Feb. 28, 2022 (WO) PCT/EP2022/054952

(51) Int. Cl. A61B 5/145; A61B 5/1455; A61B 5/00

(52) U.S. Cl. CPC A61B 5/14532; A61B 5/1455; A61B 5/7257; A61B 5/6825; A61B 5/6831

(57) ABSTRACT

A method for measuring glucose concentration in a biological material is disclosed, as well as a computer readable medium and a sensor system for performing the method. The method comprises irradiating the biological material with optical radiation having a first wavelength between 400 nanometers and 25 micrometers; detecting a first signal from the biological material at the first wavelength; irradiating the biological material with radio-frequency, RF, radiation having a second wavelength between 1 millimeter and 30 centimeters; detecting a second signal from the biological material at the second wavelength; and determining a concentration of glucose in the biological material based on the first signal and the second signal. The sensor system comprises an optical radiation source, an optical radiation detector, an RF radiation source, and RF radiation detector, and a processing device.

### CROSS REFERENCE TO RELATED APPLICATION

This application is filed under 35 U.S.C. § 111(a) and is based on and hereby claims priority under 35 U.S.C. § 120 and § 365(c) from International Application No. PCT/EP2022/054952, filed on Feb. 28, 2022, and published as WO 2022/184623 A1 on Sep. 9, 2022, which in turn claims priority from Great Britain Application No. 2102880.8, filed in the United Kingdom on Mar. 1, 2021. This application is a continuation-in-part of International Application No. PCT/EP2022/054952, which is a continuation of Great Britain Application No. 2102880.8. International Application No. PCT/EP2022/054952 is pending as of the filing date of this application, and the United States is an elected state in International Application No. PCT/EP2022/054952. This application claims the benefit under 35 U.S.C. § 119 from Great Britain Application No. 2102880.8. The disclosure of each of the foregoing documents is incorporated herein by reference.

### TECHNICAL FIELD

The present disclosure relates to devices for measuring glucose concentration in a biological material and associated methods for measuring glucose concentration in a biological material. In particular, the present disclosure relates to non-invasive measuring glucose concentration using electromagnetic radiation.

### BACKGROUND

Measuring biological parameters (e.g., glucose or other analyte concentration in blood, heart rate, blood pressure, respiration rate) of a biological material can present challenges. In general, it may require either a sample to be extracted from the biological material or an interaction between the biological material and the sensing equipment. For in vivo measurements of biological parameters, e.g., medical testing in humans, this can cause discomfort to the subject under test. Invasive techniques include taking a blood sample or putting the subject in an uncomfortable imaging system for a prolonged amount of time.

One approach to non-invasively measure biological parameters in a biological material requires detecting changes in electromagnetic radiation in the presence of a sample. This can have the advantage of improved comfort as it is possible to make sources of electromagnetic radiation on a small size. Also, the measuring can be performed from entirely outside of the body, with the electromagnetic radiation entering the body, interacting with biological material(s) therein and exiting the body to be detected. Monitoring changes in the detected radiation can be correlated to changes in the biological material and its parameters.

However, measuring biological parameters from outside the biological material by using electromagnetic radiation has limitations. For example, it is generally not usually possible to isolate any particular component or molecule of interest within the biological material. Therefore, it can be difficult to determine whether an effect on the electromagnetic radiation interacting with the biological material is caused by the component or molecule of interest, or some other component or molecule. Even if a particular molecule provides a unique signature response, it is often overshadowed by the responses from other nearby substances.

The above challenges result in poor sensitivity, specificity and consistency in detecting biological parameters in a biological material for non-invasive methods using electromagnetic radiation for measuring the biological parameter(s).

### SUMMARY

According to a first aspect, a method for measuring glucose concentration in a biological material comprises: irradiating the biological material with optical radiation having a first wavelength between 400 nanometers and 25 micrometers; detecting a first signal from the biological material at the first wavelength; irradiating the biological material with radiofrequency, RF, radiation having a second wavelength between 1 millimeter and 30 centimeters; detecting a second signal from the biological material at the second wavelength; and determining a concentration of glucose in the biological material based on the first signal and the second signal.

The first wavelength may be between 780 and 2500 nm, optionally between 1350 and 1400 nm or between 1600 and 1670 nm. The second wavelength may be less than 2 centimeters. The second wavelength may be 8.2 mm. The first wavelength may be longer than a thickness of the biological material. The second wavelength may be shorter than the thickness of the biological material.

The irradiating the biological material with optical radiation may further comprise irradiating the biological material at a third wavelength between 400 nanometers and 25 micrometers, wherein the irradiated optical radiation interaction with glucose at the third wavelength is independent of the concentration of glucose; and detecting a third signal at the optical radiation detector, wherein the determining the concentration of glucose is further based on the third signal. The third wavelength may be between 1400 and 1550 nm, optionally between 1500 and 1550 nm.

The irradiated optical radiation may be, at least in part, absorbed or scattered by an analyte at the first wavelength. The irradiated RF radiation may be, at least in part, absorbed or scattered by an analyte at the second wavelength. The first signal and/or the second signal may be a reflection signal reflected off the biological material. The first signal and/or the second signal may be a transmission signal transmitted through the biological material. The irradiating with optical radiation may be performed concurrently with the detecting the first signal. The irradiating with optical radiation may be performed sequentially with the detecting the first signal. Either way, the detected first signal is responsive to the irradiating the biological material with optical radiation. Likewise, irradiating with RF radiation may be performed concurrently with the detecting the second signal. The irradiating with RF radiation may be performed sequentially with the detecting the second signal. Either way, the detected second signal is responsive to the irradiating the biological material with RF radiation. The irradiating with optical and RF radiation, and respective detecting of the first and second signals, may be performed concurrently or sequentially. If sequentially, the interval between detecting the first signal and second signal may be less than an interval over which glucose concentration in the biological material substantially changes (to the degree of accuracy sought by the method).

The irradiated optical radiation and the first signal may comprise a first plurality of wavelengths including the first wavelength, wherein the determining the concentration of glucose is based on the first signal at each of the first plurality of wavelengths. Alternatively or additionally, the irradiated RF radiation and the second signal may comprise a second plurality of wavelengths including the second wavelength; wherein the determining the concentration of glucose is based on the second signal at each of the second plurality of wavelengths. The first plurality of wavelengths may include 1370 nm and 1630 nm.

The first signal may be detected by an optical radiation detector and the method may further include filtering optical radiation received from the biological material so that only optical radiation at the first wavelength is detected at the optical radiation detector. Alternatively or additionally, the second signal may be detected by an RF radiation detector and the method may further include filtering RF radiation received from the biological material so that only RF radiation at the second wavelength is detected at the RF radiation detector.

The method may further comprise detecting, at one or more auxiliary sensor, an auxiliary signal associated with a property of the biological material; wherein the determining the concentration of glucose is further based on the auxiliary signal; wherein the one or more auxiliary sensor comprises one or more of: an accelerometer, a strain gauge, a thermometer, a pressure sensor, an impedance sensor and an electrodermal activity sensor. For example, the auxiliary sensor may be a thermometer and the method may comprise determining the concentration of glucose in part based on the temperature of the biological material (in addition to being based on the first and second signals).

The method may comprise modulating the optical radiation, wherein the modulating comprises modulating one or more of amplitude, frequency and phase. The method may comprise: creating a fast Fourier transform (FFT) of the first signal, wherein the determining the concentration of glucose includes using the FFT of the first signal. Alternatively or additionally, the method may comprise modulating the RF radiation, wherein the modulating comprises modulating one or more of amplitude, frequency and phase. The method may comprise: creating a fast Fourier transform (FFT) of the second signal, wherein the determining the concentration of glucose includes using the FFT of the second signal.

The method may be in vivo. The biological material may be one of: a web of skin between a finger and a thumb; an earlobe; a wrist; an armpit; a lip; and a foodstuff.

The determining may comprise inputting the first and second signal into an artificial intelligence model. The artificial intelligence model may be a machine learning model, e.g., configured to use an artificial neural network. The artificial intelligence model may be a machine learning model for determining glucose concentration in the biological material based on the first signal at 1370 nm and 1630 nm and the second signal at 8.2 mm.

The method may comprise securing the biological material in a fixed position with respect to an optical radiation source and an RF radiation source, wherein the optical radiation source and the RF radiation source perform the irradiating the biological material with optical radiation and the irradiating the biological material with radio-frequency, respectively. The optical radiation source and the RF radiation source may irradiate overlapping or adjacent portions of the biological material.

The method may further comprise determining from the first signal one or more of: a value of transmittance; a value of absorptance; a value of reflectance; a value of delta transmittance/reflectance/absorptance (explained further below); a waveform or data string representing the first signal; an image of the biological material; a color or color spectrum of the biological material; and a temperature of the biological material in a sensing area. The determining the concentration of glucose may be based on any of the above parameters derived from the first signal. Accordingly, the determining being based on a parameter derived from the first signal means that the determining is based on the first signal.

The method may further comprise determining from the second signal one or more of: a value of transmittance; a value of absorptance; a value of reflectance; a value of delta transmittance/reflectance/absorptance (explained further below); a waveform or data string representing the first signal; a value of S21 s-parameter amplitude and/or phase; a value of S11 s-parameter amplitude and/or phase; a value of S22 s-parameter amplitude and/or phase; a temperature of the biological material in a sensing area; and a waveform or data string representing the second signal. The determining the concentration of glucose may be based on any of the above parameters derived from the second signal. Accordingly, the determining being based on a parameter derived from the second signal means that the determining is based on the second signal.

According to an aspect a computer readable medium comprises instructions that are executable by one or more processing device to: control an optical radiation source to irradiate a biological material with optical radiation having a first wavelength between 400 nanometers and 25 micrometers; control an RF radiation source to irradiate the biological material with radio-frequency, RF, radiation having a second wavelength between 1 millimeter and 30 centimeters; receive, from an optical radiation detector, a first signal detected by the optical radiation detector from the biological material at the first wavelength; receive, from an RF radiation detector, a second signal detected by the RF radiation detector from the biological material at the second wavelength; and determine a concentration of glucose in the biological material based on the first signal and the second signal. The computer readable medium may be either transitory or non-transitory.

The instructions may include instructions executable by the one or more processing device to perform any of the features described above with reference to methods for measuring glucose concentration in a biological material.

According to an aspect, a sensor system for measuring concentration of glucose in a biological material, the sensor comprises: an optical radiation source for irradiating the biological material with optical radiation having a first wavelength between 400 nanometers and 25 micrometers; an optical radiation detector for detecting a first signal from the biological material at the first wavelength; an RF radiation source for irradiating the biological material with radio-frequency, RF, radiation having a second wavelength between 1 millimeter and 30 centimeters; an RF radiation detector for detecting a second signal from the biological material at the second wavelength; and a processing device configured to determine the concentration of glucose in the biological material based on the first signal and the second signal.

The sensor system may be configured to perform any of the features described above with reference to methods for measuring glucose concentration in a biological material. Accordingly, the sensor system may comprise means for performing any of the features described above with reference to methods for measuring glucose concentration in a biological material.

The sensor system may comprise a holder for securing the biological material in a fixed position with respect to the optical radiation source, the optical radiation detector, the RF radiation source; and the RF radiation detector. The holder may be configured to secure the optical radiation source and the optical radiation detector on opposite sides of the biological material, wherein the first signal is a transmission signal. Alternatively or additionally, the holder may be configured to secure the RF radiation source and the RF radiation detector on opposite sides of the biological material, wherein the second signal is a transmission signal. Alternatively or additionally, the holder may be configured to secure the optical radiation source and the optical radiation detector on the same side of the biological material, wherein the first signal is a reflection signal. Alternatively or additionally, the holder may be configured to secure the RF radiation source and the RF radiation detector on the same side of the biological material, wherein the second signal is a reflection signal. The holder may comprise a wearable strap for attaching to the biological material. The wearable strap may be arranged to transmit the first signal and the second signal to a sensing device comprising the processing device. The holder may comprise a casing defining a sensing region configured to receive the biological material. The sensor system may comprise a plurality of holders, e.g., including a wearable device and a desktop device.

The sensor system may comprise one or more transmission layer positioned between a location for receiving the biological material and one or more of: the optical radiation source, the optical radiation detector, the RF radiation source, and the RF radiation detector; wherein the one or more transmission layer is an anti-reflection layer at the first wavelength or the second wavelength. Alternatively or additionally, the one or more transmission layer may be an impedance matching layer at the first wavelength or the second wavelength. The one or more transmission layers may comprise multiple layers in order to work for both the first and second wavelengths, or a range of wavelengths. The sensor system may comprise an optical filter arranged between the biological material and the optical radiation detector, wherein the optical filter is arranged to transmit the first wavelength and filter out other wavelengths.

The sensor system may comprise one or more auxiliary sensor arranged to detect an auxiliary signal associated with a property of the biological material; wherein the processing device is configured to determine the concentration of glucose based in part on the auxiliary signal; wherein the one or more auxiliary sensor comprises one or more of: an accelerometer, a strain gauge, a thermometer, a pressure sensor, an impedance sensor, and an electrodermal activity sensor. When the one or more auxiliary sensor includes an impedance sensor, the impedance sensor may comprise two or more electrodes, each arranged in contact with the biological material at a contact region; and an impedance analyzer configured to provide an electrical signal to at least one of the two or more electrodes to measure an impedance of the biological material at the contact region; wherein the auxiliary signal includes the measured impedance.

The RF radiation source and the RF radiation detector may be a single antenna. In other words, an RF antenna may be configured to both irradiate the biological material with radiofrequency, RF, radiation having a second wavelength between 1 millimeter and 30 centimeters and detect the second signal from the biological material at the second wavelength. Likewise, the optical radiation source and optical radiation detector may be a single optical device configured to irradiate the biological material with optical radiation having a first wavelength between 400 nanometers and 25 micrometers and detect a first signal from the biological material at the first wavelength. Alternatively, the optical/RF sources and detectors may be separate devices.

The sensor system may comprise a communication interface between the optical radiation detector and the processing device and between the RF radiation detector and the processing device, wherein the communication interface comprises either electronic wiring or a wireless connection.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 shows a schematic view of a sensor system for measuring glucose concentration in a biological material.

FIGS. 2A and 2B show a cross-sectional view of a holder engaging a biological material.

FIG. 3 shows a sensor device.

FIG. 4 shows a sensor device.

FIGS. 5A, 5B, 5C, 5D, 5E, 5F and 5G show a sensor device.

FIG. 6 shows a diagram of method for measuring glucose concentration in a biological material.

FIGS. 7A and 7B show graphs relating to optical transmittance of glucose.

FIG. 8 shows a graph relating to optical transmittance to glucose concentration.

FIG. 9 shows a graph relating to RF S parameters of glucose.

FIG. 10 shows a graph relating to RF S parameters to glucose concentration.

FIG. 11 shows a first table of results.

FIG. 12 shows a second table of results.

FIGS. 13A, 13B, 13C and 13D show graphs of training and validation loss for four example machine learning models.

FIGS. 14A, 14B and 14C show a third table of results.

FIG. 15 shows a fourth table of results.

FIG. 16 shows an exemplary auxiliary sensor.

FIGS. 17A and 17B show a graph relating to bioimpedance under various contact circumstances.

FIGS. 18A and 18B show an exemplary auxiliary sensor.

### DETAILED DESCRIPTION

Reference will now be made in detail to some embodiments of the invention, examples of which are illustrated in the accompanying drawings.

Overview

The present disclosure relates to methods and sensor systems that are dual-mode, i.e., use two radically different bands of the electromagnetic radiation spectrum. The combination of irradiating a biological material with radiation in these two bands, and detecting signals in the two bands, provides an increased accuracy of measuring glucose concentration in the biological material. The first band includes: visible light, having wavelengths approximately 400-780 nm; near-infrared radiation, having wavelengths 780-2500 nm; and mid-infrared radiation, having wavelengths 2.5-25 μm. Together this range of 400 nm to 25 μm is referred to herein as “optical radiation”, although more generally optical radiation also covers ultraviolet and long-infrared radiation, e.g., 100 nm to 1000 μm. The second band is radio frequency (RF) radiation, having wavelengths between 1 mm and 30 cm, which includes the millimeter wave radiation having wavelengths of approximately between 1 mm and 2 cm. Accordingly, different hardware can be used to produce the optical and RF radiation, e.g., an LED for optical radiation and an antenna for RF radiation.

Throughout the disclosure, the term “wavelength” should be understood as the wavelength of the radiation in a vacuum (having the permittivity of free-space ε0), unless stated otherwise. Accordingly, the wavelength ranges provided above correspond to frequency ranges by the inverse relationship to wavelength with proportionality factor of the speed of light in a vacuum. The optical radiation range is approximately 12-750 THz. The RF radiation range is approximately 1-300 GHz.

Determining glucose concentration based on detected signals at both optical infrared radiation and RF radiation produces increased accuracy in the determined value of the glucose concentration, i.e., a smaller error in the measurement, as explained in more detail below. The sensitivity, i.e., being able to detect lower concentrations, and the specificity, i.e., being able to tell substances apart and focus on the substance of interest, are also improved.

It is to be understood that the following description and drawings are intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described but can be practiced with modification and alteration within the scope of the appended claims. Although various features of the approach of the present disclosure have been presented separately (e.g., in separate figures), the skilled person will understand that, unless they are presented as mutually exclusive, they may each be combined with any other feature or combination of features of the present disclosure.

Sensor System

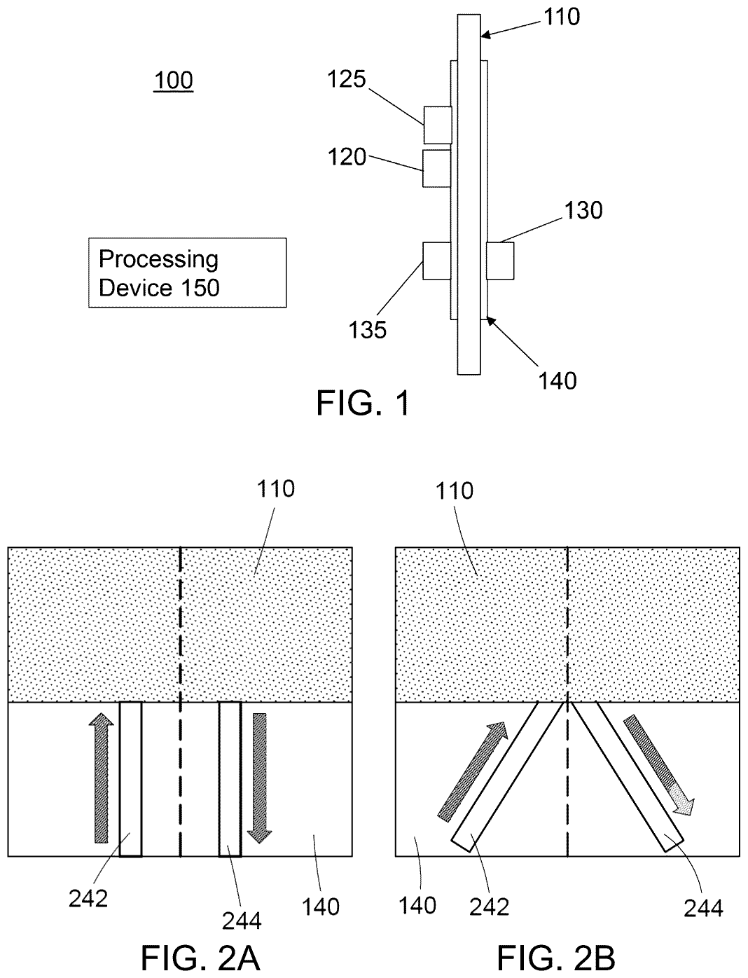

With reference to FIG. 1, a sensor system 100 suitable for measuring glucose concentration in a biological material 110 comprises components which irradiate the biological material 110 with at least two wavelengths in very different wavelength ranges. An optical source 120 is configured to irradiate the biological material with optical radiation having a first wavelength between 400 nanometers and 25 micrometers (at a frequency between 12 and 750 THz). When the optical radiation is irradiated into the biological material 110 where it interacts with or is modified by structural features or other content of the biological material. In general, the optical radiation will be influenced by tissue type of the biological material, e.g., fat layers or blood vessels, or specific molecules within the biological material, e.g., water or glucose molecules. The interaction or modification due to the biological material will cause a proportion of the optical radiation to be reflected off the biological material or components or molecules therein. Another proportion will be absorbed or scattered by the biological material, and the remainder will be transmitted through the biological material.

An optical detector 125 is positioned on the same side of the biological material into which the optical radiation was irradiated. The optical detector is configured to detect a reflected signal at the first wavelength. This reflected signal can be used to deduce glucose concentration in the biological material as it results from interaction or modification by the biological material. In alternative examples, an optical detector 125 is positioned on the side of the biological material opposite to the side into which the optical radiation is irradiated and is configured to measure a transmitted signal through the biological material. The transmitted signal can be either an attenuated direct line-of-sight signal that traverses the sample or a scattered signal that follows a complex path inside the tissue before exiting. Like the reflected signal, the transmitted signal also provides information about the glucose concentration in the biological material. Two or more optical detectors can be used, with at least one on each side of the biological material, so that both reflected and transmitted optical signals can be detected and used for determining glucose concentration.

The optical source 120 may be a light-emitting diode (LED), laser, laser diode source, or any other radiation source suitable for producing radiation at the first wavelength in the range of 400 nanometers and 25 micrometers. The optical detector 125 may be a photodetector diode, phototransistor or photoelectric cell, or any other detector suitable for detecting radiation at the first wavelength in the range of 400 nanometers and 25 micrometers.

The sensor system 100 also has an RF radiation source 130 and an RF radiation detector 135. The RF radiation source 130 is configured to irradiate the biological material with RF radiation having a second wavelength between 1 millimeter and 30 centimeters (at a frequency between 1 and 300 GHz). The RF radiation will interact or be modified by the biological material. Generally, compared to the optical radiation, the RF radiation will be influenced by bulk properties of the biological material 110 such as the dielectric permittivity of parts of the biological material, or an average or effective dielectric permittivity across the width of the biological material. For example, this would affect the amount of radiation reflected or absorbed by the biological material and/or a phase shift of the radiation as it passes through the biological material. Also, since water is highly absorbing at RF wavelengths, and particularly at millimeter wavelengths, the concentration of glucose has direct impact on the absorption caused by water since higher glucose levels mean less absorption as less water is present.

The RF radiation detector 135 is positioned on the side of the biological material opposite to the side into which the RF radiation is irradiated and is configured to measure a signal at the second wavelength transmitted through the biological material. Alternatively or additionally, an RF radiation source can be positioned on the same side as the side into which the RF radiation is irradiated in order to detect a reflection signal from the biological material.

The RF radiation source 130 and the RF radiation detector 135 are RF antennas tuned to operate in the vicinity of the biological material 110. In an arrangement, the RF radiation source 130 and the RF radiation detector 135 are a single RF antenna which is configured to both irradiate the biological material with RF radiation and to receive an RF signal (transmitted and/or reflected) from the biological material. There may be multiple RF antennas which all emit RF radiation and receive RF signals in return, or some of the RF antennas may be dedicated to irradiating only and other RF antennas dedicated to receiving reflection or transmission signals only.

A holder 140 is arranged to secure the biological material 110 in a fixed position with respect to the optical and RF radiation sources and detectors, i.e., the components which irradiate into the biological material or receive a signal from the biological material. This ensures that the radiation will be directed into the biological material 110 and that the detectors will reliably receive the transmitted or reflected signals from the biological material. The sources and detectors may be embedded in the holder 140 so that the sources irradiate directly onto the surface of the biological material and the detectors receive signals directly from the surface of the biological material. Alternatively, the source radiation may be generated away from the biological material and transmitted to the intended site of incidence, where the holder 140 directs the radiation into the biological material. Throughout the present disclosure, unless stated otherwise, the “optical radiation source” and the “RF radiation source” refer to the component that irradiates the radiation into the biological material regardless of whether or not that component generated the radiation itself from some other form of energy, or the radiation was generated elsewhere and transported to that component for irradiating into the biological material. Likewise, a detector is the component that receives a transmission or reflection signal from the biological material, even if further analysis or characterization of the signal is performed at a location away from the biological material. The optical and RF radiation sources and detectors may also comprise electronic circuitry and/or processing means for converting the radiation signals received from the biological material 110 into signals for communicating with a processing device 150. The sources and detectors may perform signal conversion, calculations or signal processing before communicating the received radiation signals, or may relay the received signals to be processed at another location.

In an example, the first wavelength is approximately 1630 nm (approximately 184 THz), the second wavelength is approximately 8.2 mm (approximately 37 GHz), together used for determining glucose concentration. For example, the first wavelength is between 1620 nm and 1640 nm, and the second wavelength is between 8.1 mm and 8.3 mm.

The holder 140 defines a sensing region configured to receive the biological material 110. This coincides with the region where the holder 140 is configured to direct the optical radiation and RF radiation, and where the holder positions the optical and RF radiation detectors. Accordingly, the sensing region is adjacent to any source and corresponding detector for receiving a reflection signal; and the sensing region is between any source and corresponding detector for receiving a transmission signal.

In alternative arrangements, the sensor system may further comprise one or more transmission layers positioned between a source and the biological material to increase the penetration of radiation into the biological material, or positioned between the biological material and a detector to increase the extraction of radiation from the biological material. This increases the strength of a corresponding received signal and improves the signal to noise ratio compared to irradiating the radiation onto the biological material with no intermediate layer. The transmission layer may be an anti-reflection coating so that irradiation from the source is substantially fully coupled into or out of the biological material and not reflected off the surface. The transmission layer may also be an impedance matching layer, which increases the penetration of radiation by reducing the boundary effects due to the difference between electromagnetic impedance (or dielectric permittivity) of the biological material and the surrounding medium. The transmission layer may also act like a lens, to focus the energy at a particular region within the biological material. The one or more transition layers may comprise a metamaterial, e.g., array of subwavelength elements arranged to resonate at the first or second wavelength of the sources and designed to provide a desired effective dielectric permittivity value or distribution. Examples of a such techniques can be found in PCT patent publications WO 2013/144,652 titled “Electromagnetic Imaging” and WO 2015/128657 titled “SENSOR”. The one or more transmission layers may be secured in place by the holder 140.

Returning to FIG. 1, the sensor system further comprises a processing device 150. The processing device is configured to determine glucose concentration in the biological material based on the signals detected by the optical radiation detector 125 and the RF radiation detector 135. The processing device 150 is in communication with the detectors 125/135 in order to receive the detected signals. The processing device may also be configured to control the irradiation of the biological material 110 by the optical radiation source 120 and RF radiation source 130, e.g., setting the first and second wavelengths, the irradiated power from each source, modulation of the source radiation, etc. The communication between the processing device 150 and other components of the sensor system 100 may be wireless communication or through electrical connections. For example, a communication interface that includes a wireless connection is coupled between the RF radiation detector 135 and the processing device 150. The sensor system may further comprise a memory for storing readable instructions for determining glucose concentration based on the signals from the detectors or other actions performed by the processing device 150. The memory may also store previous signals from the detectors.

In alternative arrangements to the sensor system of FIG. 1, the sensor system may comprise multiple optical sources 120 and/or multiple RF radiation sources 130. This can increase the area of the biological material 110 which is measured, or increase the signal-to-noise ratio by using a higher intensity of radiation to be transmitted and detected.

In some arrangements, the sensor system comprises an optical filter positioned between the biological material and the optical radiation detector. The optical filter is arranged to transmit the first wavelength and filter out other wavelengths. This means that only optical radiation at the first wavelength is detected at the optical radiation detector for the first signal. This can be done using narrowband notch filters, e.g., placed at the entrance of the optical radiation detector, so that only light that is emitted by the optical radiation source is detected at the corresponding detector. Any stray or background light at other wavelengths is filtered out and does not become part of the detected first signal. This improves the signal-to-noise ratio of the first signal. Alternatively or additionally, the sensor system further comprises an RF filter positioned between the biological material and the RF radiation detector. The RF filter is arranged to transmit the second wavelength and filter out other wavelengths. This means that only RF radiation at the second wavelength is detected at the RF radiation detector for the first signal, with the corresponding advantages for the second signal as explained above for the first signal.

In some arrangements, the sensor system further comprises an auxiliary sensor or sensors including one or more of: an accelerometer or strain gauge (to detect motion); an infrared or contact thermometer (to detect tissue/sample temperature); a pressure sensor (to detect applied pressure to the sample and optimize clamping); and an impedance or EDA (electrodermal activity) sensor. The auxiliary sensor is arranged to detect one or more properties of the biological material 110 which could in theory influence the detected signals, such as motion, temperature, strain, EDA, etc. The processing device 150 may further base the determination of glucose concentration based on an auxiliary signal from the auxiliary sensor or auxiliary sensors.

With reference to FIGS. 2A and 2B, the holder 140 may direct radiation from the optical source 120 and/or the RF radiation source 130 at an angle of incidence into the biological material. In this arrangement, the holder 140 comprises a transmitting waveguide 242 for transmitting radiation from the respective source 120, 130 into the biological material and a receiving waveguide 244 for receiving radiation from the biological material to the respective detector 125/135. With reference to FIG. 2A, the transmitting waveguide 242 and the receiving waveguide 244 are substantially parallel. This arrangement will detect diffuse radiation that has penetrated the biological material and interacted with structural components or molecules therein. With reference to FIG. 2B, the transmitting waveguide 242 and the receiving waveguide 244 are arranged at an acute angle with respect to the surface of the holder 140 which faces the biological material 110, e.g., a surface of the holder which contacts the biological material. This causes the transmitted radiation to impinge the biological material at an angle of incidence greater than zero degrees. The transmitting and receiving waveguides are arranged at approximately the same angle with respect to the surface of the holder 140. This arrangement will detect scattered radiation from within the biological material. Accordingly, either the parallel or angle waveguide arrangements can be chosen depending on the particular interaction of the radiation with the biological material of interest, i.e., which will provide the most accurate or reliable measurement of glucose concentration.

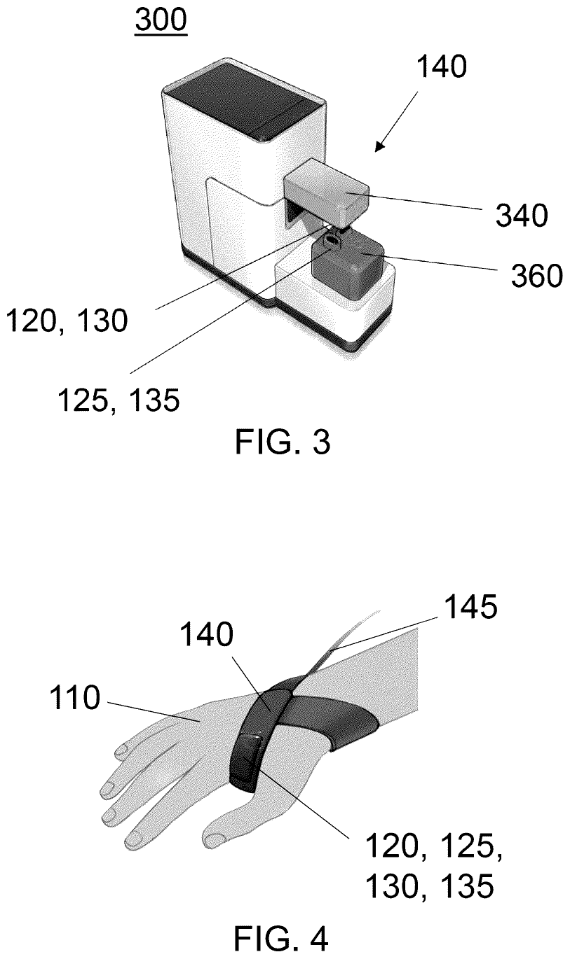

With reference to FIG. 3, an arrangement of the sensor system comprises a sensing device 300. The sensing device 300 comprises a casing which contains the sensor system components explained above with reference to FIG. 1. The holder 140 of the sensing device comprises an arm 340 and a platform 360 which, in use, secure a biological material between the arm and the platform in a sensing region. The arm 340 and platform 360 comprise the sources and detectors of the sensing device 300. The optical source 120 and RF radiation source 130 are held by the arm 340 and the optical detector 125 and RF radiation detector 135 are held by the platform 360. In alternative arrangements, the sources may be located on the platform 360 instead and the detectors on the slidable arm 340, or all on the slidable arm 340, or all on the platform 360, or any combination of on the slidable arm 340 and platform 360 in accordance with the requirements of the application.

In use, the biological material 110 is positioned on the platform 360. The arm 340 is arranged to slide towards the platform 360 to engage the biological material. This can be done either manually by a user or controlled by the sensing device 300, e.g., by a processing device 150. The arm 340 is slidably attached to the rest of the casing, e.g., by a rail. The arm 340 and platform 360 are arranged to pinch the biological material in the sensing region such that the radiation sources 120, 130 can irradiate the biological material 110 and detectors 125, 135 can receive signals from, the biological material 110.

In some arrangements, the arm 340 or the platform 360 comprises a pressure sensor arranged to detect the pressure exerted on the biological material. This can be used as feedback for controlling the arm 340 to determine when it has slid far enough to engage the biological material but not to damage the biological material, e.g., hurt or injure a person being tested by the sensing device.

In some arrangements, the arm 340 and platform 360 are detachable from the rest of the casing in order to increase the mobility and versatility of the sensing device 300. The rest of the casing therefore is a dock. In this arrangement, the arm 340, platform 360, and the dock each comprise a communication module to communicate instructions and signals therebetween. The communication may be wired such as via a USB cable. Alternatively, the communication may be wireless, e.g., using Bluetooth or other short-range communication protocols. When the sensors and detectors are positioned on a biological material, the dock is arranged to send instructions to the sensors and detectors, and to receive the detected first and second signals from the biological material. The dock may be arranged to charge batteries in the arm 340 and platform 360 when they are docked.

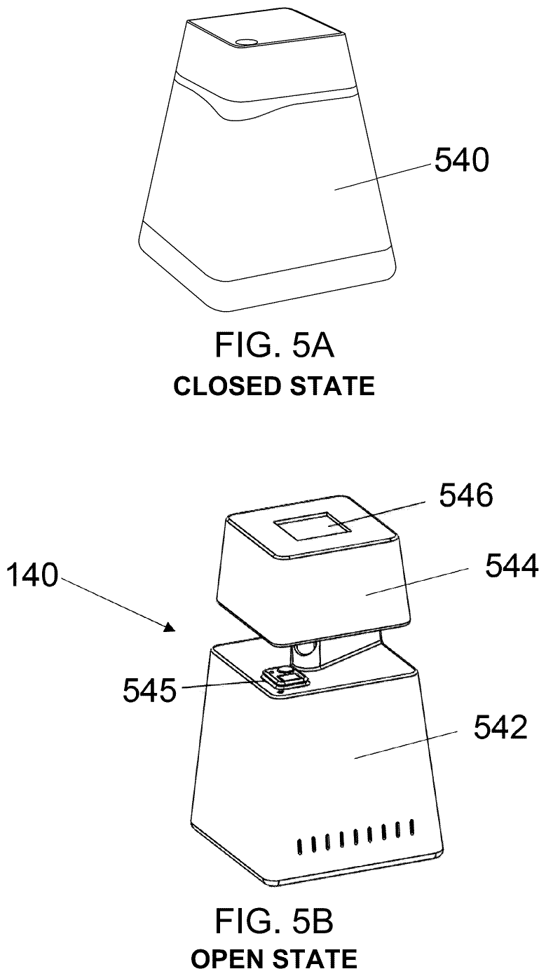

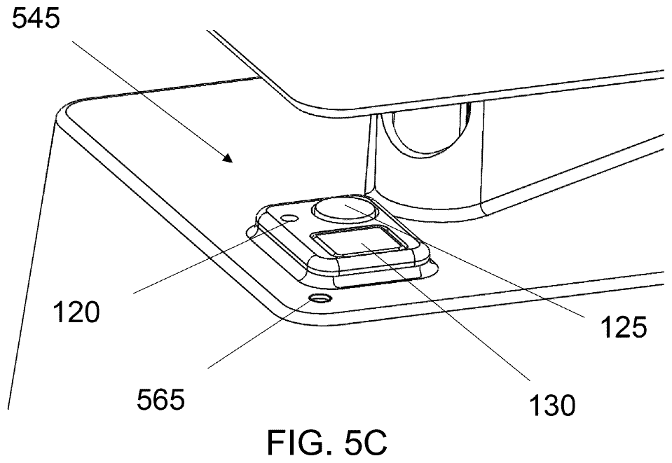

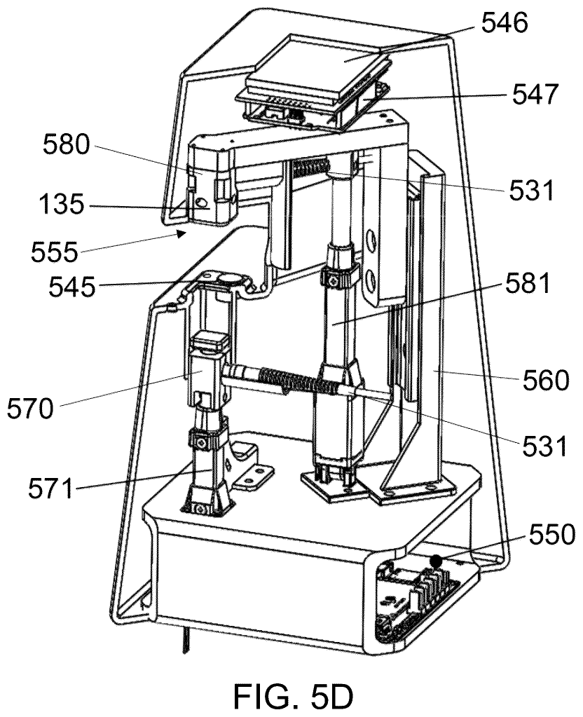

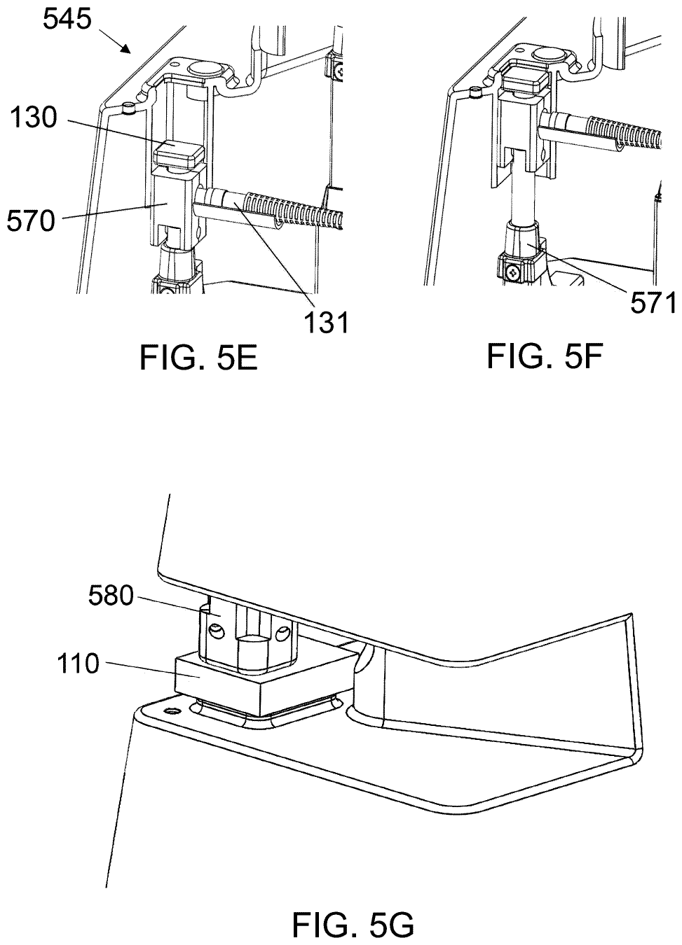

With reference to FIG. 4, in an arrangement of the sensor system, the holder 140 is a wearable strap for attaching to a hand, i.e., a biological material 110. The wearable strap comprises the optical source 120, the optical detector 125, the RF radiation source 130, and the RF radiation detector 135 arranged such that, when the wearable strap wrapped around the hand, the sensors and detectors engage a web of skin between thumb and forefinger of the hand in a sensing region between sources and detectors. The wearable strap is arranged to attach to the hand by forming a loop between the thumb and forefinger and a loop around the wrist, with an end of the wearable strap arranged to attach to an intermediate portion of the wearable strap. The attachment may be by hook-and-loop fastener (such as Velcro™), buttons, poppers, etc. A cable 145 is attached to the wearable strap for communication with another device and/or to provide power to the wearable strap components. In an example, the wearable strap is arranged to communicate with a sensing device 300 as described with reference to FIG. 3 or FIG. 5. The wearable strap may contain its own processing device or communicate with the sensing device to use the processing device of the sensing device. An advantage of arrangements wherein the holder 140 is the wearable strap is that the irradiation and detection of radiation can be performed with increased comfort to the person being tested by the device. For example, the wearable strap can be worn during sleep, with repeated or continual readings being taken and communicated back to the sensing device.

In some arrangements, instead of a hand, the wearable strap is arranged to attach to another body part. In some arrangements, instead of a wearable strap, the holder comprises a clip arranged to attach to a web of skin between a finger and a human earlobe, an armpit or a lip. In some arrangements, instead of a wired connection using a cable 145, the wearable strap is configured to communicate with the other device wirelessly, e.g., using Bluetooth or other short-range communication protocols.

The sensing device 300 and/or the wearable strap as described above with reference to FIGS. 3 and 4 may further comprise a display screen for displaying a value or range of values for glucose concentration as determined by the processing device 150 based on the first and second signals. The sensing device 300 and/or the wearable strap may further comprise a user interface for receiving user inputs to control the operation of the sensing.

With reference to FIGS. 5A to 5G, in an arrangement a casing of the sensing device 300 has a frustrated square-based pyramid shape 540. In other arrangements, the casing has a different shape, such as cylindrical, oblong, conical, irregular, etc. The casing comprises a first portion 542 and a second portion 544. The second portion 544 of the casing is arranged to slide towards and away from the first portion 542 of the casing, e.g., by being slidably attached to the first portion 542 via a pillar 560. A sensing region is provided between the first and second portions, with the optical source 120, optical detector 125 and a first RF antenna, for functioning as an RF radiation source 130, supported by the first portion 542 at a first sensing area 545. The first sensing area 545 also comprises a temperature sensor 565 as an auxiliary sensor. A second RF antenna, for functioning as an RF radiation detector 135, is supported by the second portion 544 at a second sensing area 555. As previously explained, RF antennas may act either as an RF radiation source or as an RF radiation detector and any single RF antenna may function as both. Therefore, while FIG. 5 shows the RF radiation source 130 being in the first sensing area 545 and the RF radiation detector 135 being in the second sensing area 555, in alternative arrangements these may be the other way around, or more generally both RF antennas function as both source and detector.

With reference to FIG. 5D, the sensing device comprises a first RF antenna support 570 for supporting the first RF antenna 130 and a second RF antenna support 580 for supporting the second RF antenna 135. The sensing device comprises a first actuator 571 in the first portion 542 for extending and retracting the first RF antenna 130 towards or away from the sensing region and a second actuator 581 in the second portion 544 for extending and retracting the second RF antenna 135 towards or away from the sensing region. RF cables 531 are connected to the first and second RF antennas to transmit RF signals to and from the first and second RF antennas, i.e., to transmit RF radiation to be irradiated by the RF radiation source and to receive the second signal from the RF radiation detector.

The sensing device comprises a control board 550 comprising the processing device 150 and any other circuitry for powering or controlling the electronic components of the sensing device such as the sources, detectors and actuators. The control board may also comprise an RF radiation generator to generate RF radiation to be transmitted by the RF cables to the RF radiation source for irradiation. The sensing device also comprises a dedicated screen electronics board 547 for controlling the output and parameters of the screen 546. In alterative arrangements, the sensing device may have more or fewer control boards located throughout the sensing device or may communicate with another device comprising control boards for controlling the components of the sensing device.

With reference to FIGS. 5E, 5F and 5G, when a biological material 110, such as a hand, is positioned between the first and second portions, the second portion 544 is arranged to slide towards the first portion 542 until the sources and detectors engage the biological material 110. The first actuator is arranged to extend the first RF antenna support 570 and first RF antenna 130 from a retracted position (e.g., as shown in FIG. 5E) towards the sensing region to an extended position (e.g., as shown in FIG. 5F). The second actuator is arranged to extend the second RF antenna support 580 and second RF antenna 135 towards the sensing region to an extended position (e.g., as shown in FIG. 5G) and to engage the biological material 110. This provides a reliable and reproducible contact between the RF antennas and the biological material and correspondingly increases the accuracy of the second signal detection. The sensing device may further comprise a pressure sensor to detect the pressure applied to the biological material by the holder mechanism comprising the first and second actuators and RF antenna supports. The feedback from the pressure sensor can be used by the control board 550 (e.g., processing device 150) to prevent damaging or injuring the biological material 110 or providing discomfort for the person being tested.

In some arrangements, the optical source and/or optical detector have its own actuation mechanism, e.g., according to the mechanism described above with reference to FIGS. 5E, 5F and 5G, either instead of or in addition to the actuators arranged to actuate the RF antennas.

Apart from the shape and holder mechanism for engaging the biological material, the sensor device 300 may have any of the features of the sensing device described above with reference to FIGS. 3 and 4.

Measuring Glucose Concentration in a Biological Material

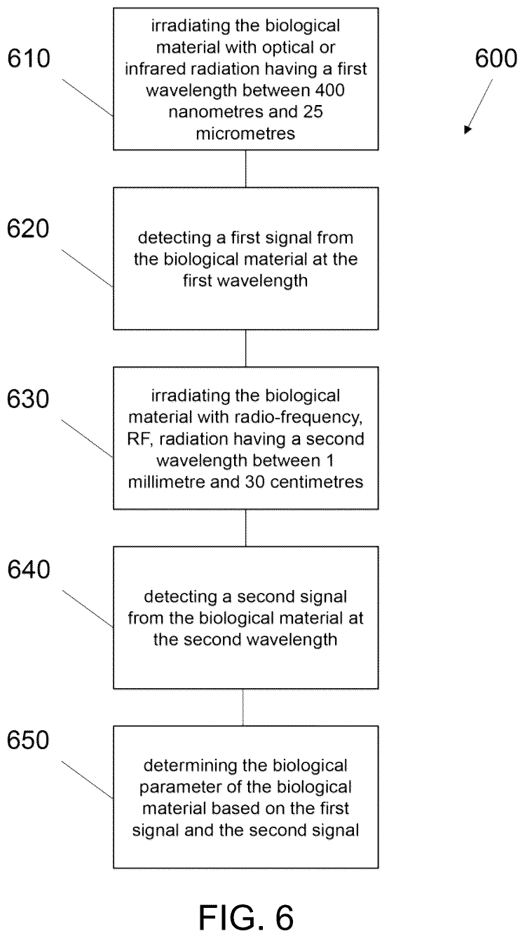

With reference to FIG. 6, a method 600 for measuring glucose concentration in a biological material comprises irradiating 610 the biological material with optical radiation at a first wavelength between 400 nanometers and 25 micrometers, that is, between 12-750 THz in frequency. The method further comprises detecting 620 a first signal from the biological material at the first wavelength. The method further comprises irradiating 630 the biological material with radio-frequency, RF, radiation having a second wavelength between 1 millimeter and 30 centimeters, that is, a frequency of 1-300 GHz. The method further comprises detecting 640 a second signal from the biological material at the second wavelength. The method 600 further comprises determining 650 a biological parameter, which is glucose concentration, in the biological material based on the first signal and the second signal.

In general, the determining 650 glucose concentration means providing a value for glucose concentration in the biological material under test, e.g., determining that a glucose concentration in blood is 100 milligram per deciliter (mg/dl). In practice, the value is of course an approximate or estimated value, which may be expressed as a value and an estimated uncertainty or error bars.

In an example, the method further comprises providing the biological material to be analyzed and securing the biological material in a fixed position with respect to the sources and detectors, for example using a holder 140 as described above.

The irradiating 610 with optical radiation and/or the irradiating 630 with RF radiation may include controlling a corresponding optical radiation source 120 and/or RF radiation source 130 to irradiate the biological material at their respective wavelengths. The irradiating with radiation at the first and second wavelengths may be performed simultaneously or sequentially. The irradiating and detecting at the first and second wavelengths for determining the glucose concentration should normally be performed within a timescale over which glucose concentration is unlikely to change dramatically.

In an example, the irradiating 610 with optical radiation comprises irradiating the biological material with a first plurality of wavelengths including the first wavelength. The plurality of wavelengths can be in a continuous range of wavelengths, or at discrete wavelengths. In this example, the first signal may include a multiple components corresponding to the detected signals at each wavelength of the first plurality of wavelengths, or the first signal may be a combination of the signals at the first plurality of wavelengths. As an example, the first plurality of wavelengths comprises 1370 nm (approximately 219 THz) and 1630 nm (approximately 184 THz) and glucose concentration is based on the detected first signal at both of these wavelengths.

Additionally or alternatively, the irradiating 630 of RF radiation comprises irradiating the biological material with a second plurality of wavelengths including the second wavelength and same principles apply to the corresponding detecting 640 and the second signal. By using a plurality of wavelengths, a more detailed or more accurate determination of glucose concentration can be made.

In general, the optical radiation and the RF radiation will penetrate the surface of the biological material and interact with the structure of molecules of the biological material such that the signal at the corresponding detector will be dependent on the concentration of glucose in the biological material. The method may further comprise focusing and/or coupling the radiation into the biological material using one or more transmission layers or other elements and previously described. The transmission layers may comprise one or more of focusing or defocusing lenses, lens tubes, anti-reflection films, diffusers, or skin impedance matching films.

The first and/or second signals can each be either a reflection signal or a transmission signal as previously described. Reflection signals indicate how much radiation the biological material has reflected at the wavelength irradiated which depend on the concentration of glucose, and may also depend on, for example, the absorption spectrum of the biological material, or a size of a step in dielectric permittivity between two components of the biological material, etc. A transmission signal may depend on any or all of the same factors. If both a reflection and transmission signal are detected, then in combination the difference between the power irradiated and the power detected as either reflection and transmission will approximate the amount of power absorbed or scattered by the biological material during irradiation. The signals can also be used to determine a difference in intensity and/or amplitude and/or phase and/or polarization between the input and output radiation, which is affected by the absorption, reflection and transmission characteristics of the biological material. The absorption characteristics of the biological material indicates the presence or concentration of glucose in the biological material. Additionally, the absorption characteristics may indicate the composition of the biological material such as fat content, water content, etc.

In an example, the method further comprises modulating either or both of the optical radiation and the RF radiation. This may be done by controlling the optical radiation source or the RF radiation source to modulate one or more of the amplitude, frequency and phase of the radiation. This can be used to improve the signal-to-noise ratio of detection and so improve precision of detecting the first and second signals. The frequency of the modulation may be between 100 Hz and 1 GHz, and, as an example, 1 kHz. For example, the optical source or RF radiation source may modulate amplitude of the radiation in periodic manner, increasing and decreasing at a constant rate according to the modulation frequency. The corresponding detector will then detect a modulated signal which can be distinguished from background radiation entering the biological material from ambient sources. The differences in the amplitude of the received modulated signal can be correlated to changes in the biological material. This will then remove a source of error in the determination of glucose concentration based on the first and second signals. In another example, the frequency is modulated, which will also modulate the first or second wavelength over a range of wavelengths. A timing reference can be sent to the corresponding detector so that the source and detector are synced and the detector will detect the signal at the corresponding modulated frequency in time with the source. This could be achieved using a variable radiation filter, or by detecting signals at a range of frequencies and selecting the signal which follows the modulated frequency. In another example, a modulated phase delay is applied to the radiation from a source, or a phase difference between two sources is modulated and the modulated signal is detected at the corresponding detector.

In examples where one or more of the radiation sources are modulated, the processor may perform a fast Fourier transform (FFT) on the detected signals, i.e., create an FFT of the first signal, second signal, or both. This is one example how to detect changes in the modulated signal. The FFT of the detected signal(s) provide a representation of the frequency composition of the signal and a peak of the FFT at the modulation frequency indicates the component of the detected signal which is modulated at the modulation frequency. Accordingly, the parameters of this peak of the FFT contains information about the part of the detected signal due specifically to the modulated radiation source. Therefore, the FFT of the first or second signal can be used in the determining of glucose concentration.

In general, the first and second wavelengths of radiation (or first and second pluralities of wavelengths) to be used are chosen according the particular characteristics of glucose. For example, the first and second wavelengths may be chosen to be in a range of wavelengths at which radiation is sensitive to the concentration of glucose. The first and second wavelengths may also be chosen based at least in part on a dimension of the biological material to be analyzed. For example, the second wavelength, i.e., the wavelength of the RF radiation, is chosen to be greater than a thickness of the biological material to be analyzed. In such examples, the RF radiation experiences the biological material as a subwavelength object and this determines the nature of the interaction, as the radiation will experience the bulk properties of the biological material. Conversely, the RF radiation will not interact directly with individual structures or molecules within the biological material. Conversely, the first wavelength, i.e., the wavelength of the optical radiation may be chosen to be less than the thickness of the biological material to be analyzed. This radiation will interact with objects on a smaller scale and in general will be more sensitive to the particular composition of biological material, how much of different molecules or cell types are present, rather than just the bulk characteristics.

Using radiation from both the optical part of the electromagnetic spectrum, in particular between 400 nanometers and 25 micrometers, and the RF radiation part of the spectrum, between 1 millimeter and 30 centimeters, to determine the glucose concentration will improve the accuracy, sensitivity and/or specificity of the determination. For optical radiation, there may be multiple signals from a biological material that will overlap, such as glucose, hemoglobin, lipids, proteins, and water. In addition, external factors such as the condition of the skin, dirt, thermal noise, sample temperature variations, and motion of the sampling area can in principle affect the first signal at the first wavelength in the optical range. This could make it difficult to provide specificity, i.e., be sure that the changes in the optical signal are due to the glucose concentration and not due to these external factors or other substances in the biological material that are not of interest.

On the other hand, RF radiation is less susceptible to external factors that are much smaller than the second wavelength, as it tends to sample the whole volume under investigation. As an example, changes in the spatial distribution of water inside the sample has a small effect to the second signal at an RF radiation wavelength but can dramatically change the first signal at an optical wavelength (which is very depth-dependent). However, RF radiation signals are usually not specific enough to identify glucose or determine glucose concentration. Rather, they typically track more accurately the changes when the rest of the biological material constituents do not change more significantly than the glucose concentration.

Accordingly, determining the glucose concentration based on both the first signal at the optical first wavelength and the second signal at the RF radiation second wavelength pinpoints the glucose concentration based on both the more sensitive or noisy (with respect to glucose concentration) first signal and the bulk-property dependent second signal. In essence, the bulk-property information of the second signal can be used to clean and/or improve the accuracy of the first, molecule-sensitive signal, which would otherwise not be identified clearly. Accordingly, if the first signal is resonantly sensitive to glucose molecules, (e.g., in via Raman spectroscopy method), a change in that signal could be caused either by a change in the glucose concentration, or by a change in the surrounding water distribution. Since the second signal can detect and isolate the surrounding water distribution changes, this information can then be used to remove that effect from the first signal, identifying he change due to glucose concentration changes only.

This combination of optical radiation and RF radiation synergistically provides a particularly precise targeting of glucose concentration with improved accuracy compared to using only one of the two radiation ranges or using them independently to create separate measurements of glucose concentration.

Further, in arrangements where the biological material is determined using artificial intelligence, e.g., a machine learning or deep learning model, the determination using each of the optical first signal or the RF radiation second signal has a degree of uncertainty from the output of the determined value of the glucose concentration. When both signals are used, the uncertainty is reduced, e.g., by focusing on where the ranges of uncertainty from each individual readings overlap. For example, when both detector signals are used and therefore two prediction intervals can be obtained, a third combined prediction interval can be constructed. The combined prediction interval consists of the predicted values that exist in both individual sensor uncertainty intervals. This combined interval is smaller than the two original intervals, and thus increases the accuracy of the sensing system overall due to the smaller uncertainty.

The glucose concentration being “based on” the first and second signals does not mean that the determination is exclusively based on these two signals (although that is a possibility). In some examples, the determination of glucose concentration is based on one or more additional signals from an auxiliary sensor. For example, an auxiliary sensor may detect the motion the biological material (by an accelerometer), temperature (by a thermometer), electrical impedance, etc. These additional inputs to the determination may further improve the accuracy of the determination of the glucose concentration.

In an example, the glucose concentration is determined using artificial intelligence (AI), such as by inputting the first and second signals into a machine learning model trained to determine glucose concentration. For example, an artificial neural network can be used as the machine learning model to determine glucose concentration. The machine learning model can be trained using training data comprising acquired data of the first and second signals from when a biological material having a known value of glucose concentration is irradiated with optical radiation and RF radiation. In an example, the training data may comprise a value for transmittance determined from the first signal (for the optical radiation) and a value of S21 s-parameter amplitude determined from the second signal (for the RF radiation) for each known value of glucose concentration in the biological material. Alternatively or additionally, the training data may comprise one or more of the following parameters determined for known values of glucose concentration in a biological material: a value of delta transmittance determined from the first signal (explained further below); a value of S11 s-parameter amplitude and/or phase determined from the second signal; a value of S22 s-parameter amplitude and/or phase determined from the second signal; temperature of the biological material in a sensing area; a waveform or data string representing the first signal; a waveform or data string representing the second signal; parameters of the optical radiation input to the biological material; and parameters of the RF radiation input to the biological material. The training data may also include multiple data points of any of these parameters for each of a plurality of wavelengths. The training data may also include delta values comparing any of the above parameters against a reference sample other than the biological material, e.g., another body part, or against a reference wavelength which is not glucose-dependent (such as between 1400 and 1550 nm, or between 1500 and 1550 nm). Any of the inputs to the AI model, explained further below, can be included in the training data.

The training data may be acquired by using alternative methods to determine the glucose concentration in the biological material, such as blood tests or imaging, and then irradiating the same biological material with the radiation to detect the first and second signals. The machine learning model is trained to minimize the difference between the output of the model for determined glucose concentration and the “actual” or reference glucose concentration as appears in the training data (“ground truth”). When the machine learning model is applied to new inputs of the first and second signals, the machine learning model will output a value of the glucose concentration based on the first and second signal.

More specifically, as an example, for the development of the artificial neural network (ANN), the entire dataset acquired through experiments is divided into a training set and a test set. The training set typically consists of 80% of the data while the generalization of the models (validation) is tested using the remaining 20%. It is beneficial in such models that the test data is contained within the training data extremes, since ANNs are known to lose accuracy while extrapolating. The steps for development of the ANN can include:
1. segregating training and test datasets;
2. selecting an ANN architecture using k-fold cross validation;
3. selecting training termination criteria and training; and
4. validating the computational models using test dataset.

The ANNs can be developed in programming language such as Python using dedicated machine learning algorithms such as TensorFlow.

Further according to this example, out of, for example, 134 data points available from the experiments, 114 can be used for training the ANN while 20 can be reserved for validation at a later stage. For the selection of the ANN architecture (number of hidden layers and the number of neurons in every layer), a method known as k-fold cross validation can be used. In this method, the training dataset is further divided into a number of smaller datasets. Each of these are turn-wise reserved for testing, while an ANN trains using the other datasets. For example, the training dataset can be divided into 5 smaller sets. Different network architectures can then be trialed and the best architecture containing a single hidden layer with 64 neurons will be chosen for the model development.

It is advantageous if the computational models are not overfitted (test error higher than training error). For this, the termination criteria for the training may be chosen accordingly. For example, when the average validation error is observed to go beyond the training error, the training is immediately terminated. Additionally, the training will also be terminated if there is no significant improvement observed in the error on continued iterations.

In some examples, the glucose concentration is determined based on the first and second signal using a reference such as look-up tables. The look-up table takes two inputs corresponding to the first signal and second signal and determines a value of the glucose concentration. The inputs corresponding to the first and second signals may be a proportion of the irradiated radiation reflected by the biological material, or a proportion of the irradiated radiation that is transmitted by the biological material. In some examples, the glucose concentration is determined based on the first and second signal using analytical techniques, for example an equation relating two inputs corresponding to the first and second signals to a value of glucose concentration. In general, the glucose concentration may be determined using a combination of some or all of the above determination methods.

The method 600 as described above may be implemented by a sensor system 100 as described with reference to FIGS. 1 and 2 or a sensing device 300 as described above with reference to FIGS. 3 to 5.

Glucose Monitoring

The following describes further examples of the method 600 for measuring glucose concentration described above with reference to FIG. 6.

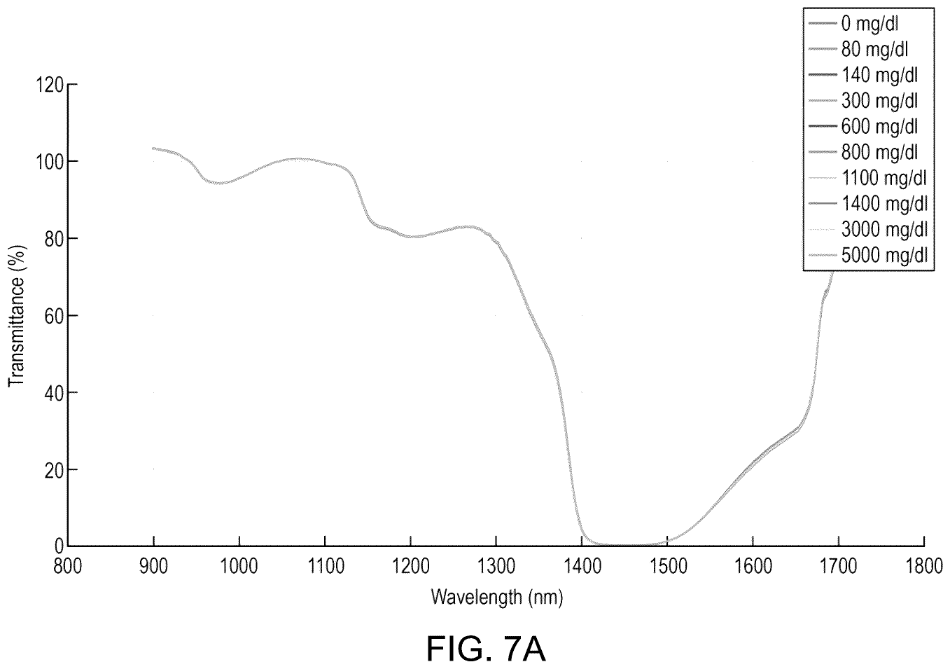

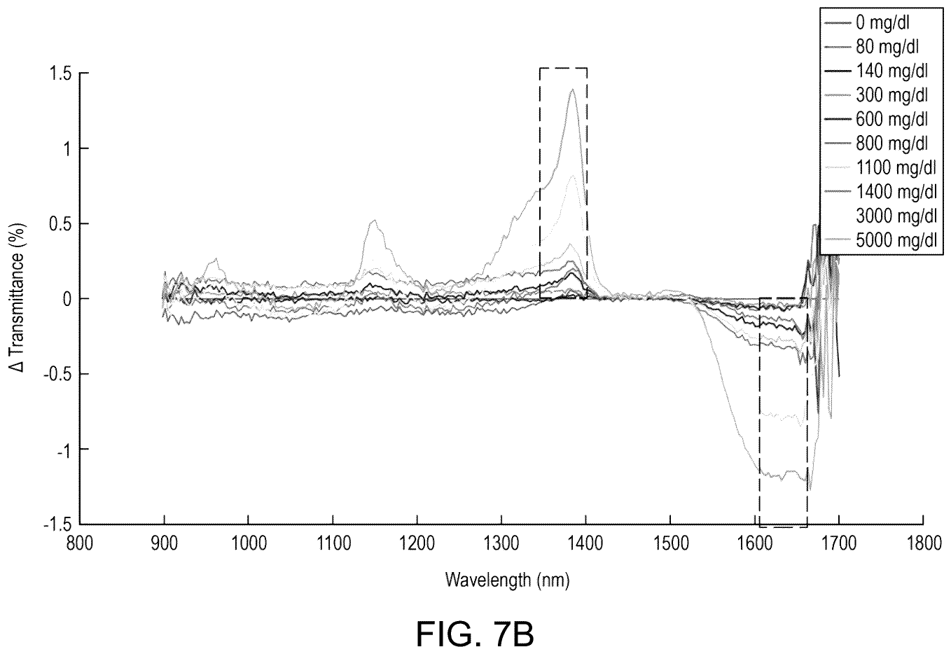

FIG. 7A shows the transmittance between 0 and 100 (y-axis) versus wavelength in between 800 nm and 1800 nm (x-axis), i.e., part of the optical range of wavelengths and, in particular, near-infrared. Transmittance is the ratio (expressed as a percentage in FIG. 7A, but sometimes also measured in dB) between radiation irradiated into the material (e.g., input power) and radiation transmitted (e.g., transmitted power). There are multiple plots of the transmittance for different concentrations of glucose dissolved in water, from 0 mg/dl to 5000 mg/dl. The transmittance values are similar across the range of plotted glucose concentrations, having close to the same percentage transmittance value as water at each wavelength. However, there are variations in the transmittance value which is shown by a delta percentage transmittance (Δtransmittance) graph as shown in FIG. 7B. FIG. 7B shows the delta percentage transmittance, which is the difference in percentage points between the transmittance values of a particular concentration of glucose and a reference concentration of 0 mg/dl glucose. The range of wavelengths along the x-axis, and the range of concentrations of glucose of the different plots are the same as for FIG. 7A. With reference to FIG. 7B, the different concentrations of glucose in a biological material are determinable based on the transmittance of radiation through the biological material. The transmittance is calculated from the first signal received at the optical detector by comparison to the radiation irradiated into the biological material by the optical radiation source. The delta transmittance can be determined for each frequency by calibrating the measured transmittance at a particular concentration against the transmittance of water or the system without a sample, and subtracting the transmittance value of water. As can be seen from FIG. 7B, the greatest differentiation between glucose concentration levels occurs in the ranges 1340 nm to 1400 nm (approximately between 214 and 224 THz) and 1550 nm to 1670 nm (approximately 179 to 193 THz). These ranges are therefore particularly effective for determining the concentration of glucose, as the transmittance is more sensitive to glucose concentration in these wavelength ranges. However, other near-infrared wavelengths in the range 780 nm to 2500 nm are also suitable (approximately 120 to 380 THz using two significant figures) and likewise optical wavelengths in the range 400 nm to 25 μm (approximately 12 THz to 750 THz, using two significant figures).

In an example, the first wavelength is between 400 nm and 25 μm, excluding 1440 nm to 1520 nm (approximately 197 to 208 THz). In other words, the first wavelength being between 400 nm and 1440 nm or 1520 nm and 25 μm. With reference to FIGS. 7A and 7B, transmittance in the range 1440 to 1520 nm is substantially independent of concentration of glucose. With reference to FIG. 7A, this corresponds to the wavelengths for which there is approximately zero transmittance for all glucose concentration levels. However, this independence can be used to improve the accuracy of the determination of the glucose concentration. Accordingly, in an example, the method further comprises irradiating the biological material at third wavelength between 1400 and 1550, or in the specific ranges 1440 to 1520 nm or 1500 to 1550 nm, wherein the radiation interaction with glucose is substantially independent of the concentration of glucose. When this radiation is detected at the optical detector, this third signal can be used as a calibration for the first signal at the first wavelength. This is because the third signal is independent of glucose concentration and so provides a reading of the transmittance due to biological material other than glucose, and any ambient or stray radiation not from the optical source. The determination of glucose concentration may therefore be further based on the third signal, to improve accuracy further.

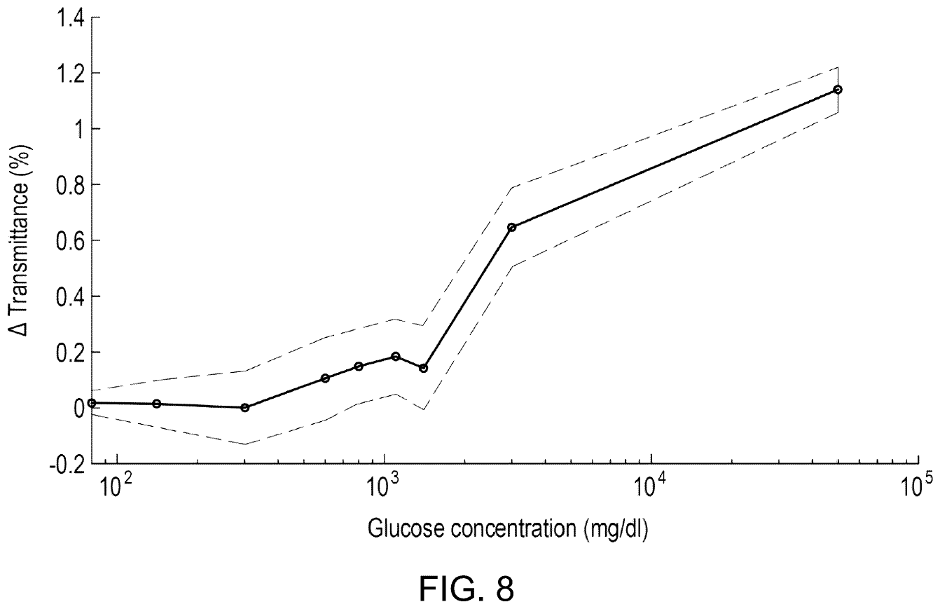

With reference to FIG. 8, the A transmittance, expressed as a percentage difference from zero concentration transmittance, is plotted against glucose concentration. Therefore, starting from a reading of transmittance, based on the first signal, a value for glucose concentration can be determined. However, using optical radiation alone is susceptible to errors due to the sensitivity of optical radiation to water or other substances.

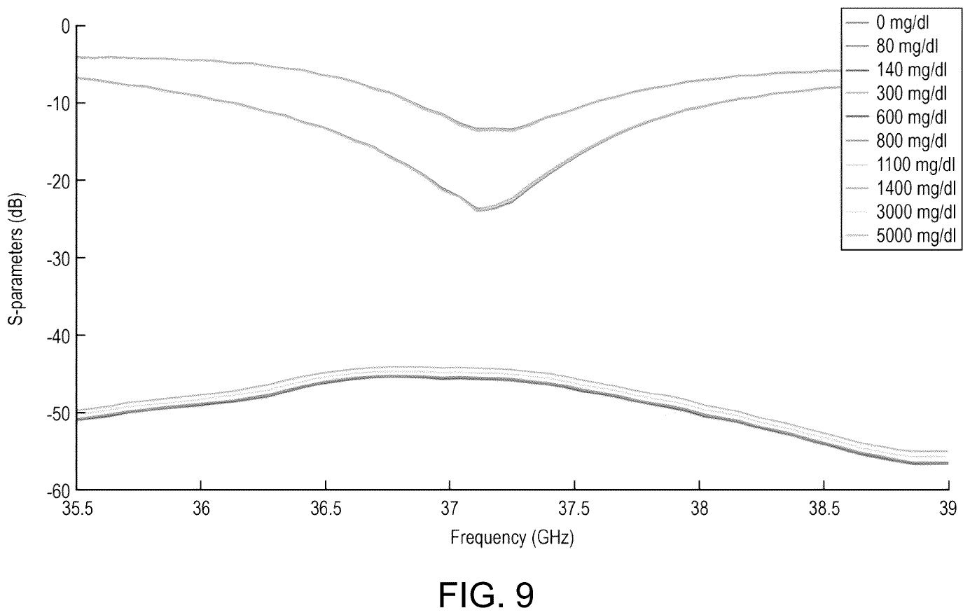

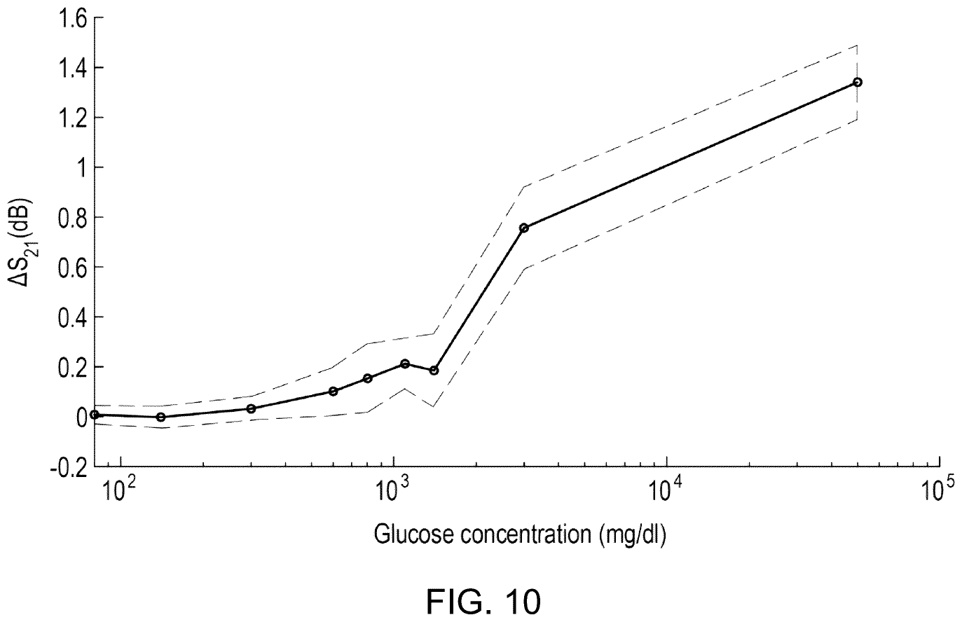

With reference to FIG. 9, the S parameters for transmission S21 and for reflection S11 (at the input side) and S22 (at the output side), which are measured ratios of transmitted or reflected radiation power to the input RF radiation power. The lower plot in FIG. 9 corresponds to the S21 parameter, the lower plot of the upper pair of plots corresponds to S22 and the upper plot of the upper pair of plots corresponds to S11. The values for the S parameters in dB are plotted for glucose concentrations ranging between 0 mg/dl to 5000 mg/dl across a frequency range of 35.5 GHz to 39 GHz (approximately 7.7 mm to 8.4 mm in wavelength). The variations in the S parameters between different concentrations of glucose can be used to determine a value for glucose concentration. With reference to FIG. 10, a graph for delta S21, i.e., the ratio of the transmission S parameter S21 at a particular concentration of glucose compared to S21 at zero concentration is plotted (in dB) for a range of concentrations (in mg/dl). With reference to FIG. 10, in a corresponding manner to FIG. 8, starting from a reading of S21, based on the second signal, a value of glucose concentration can be determined. However, using RF radiation alone is susceptible to errors due to RF radiation being affected by more parameters than just glucose concentration specifically.

When used in combination, using both optical radiation and RF radiation in the determination of glucose concentration provides an improved accuracy by mitigating the limitations of each range of wavelengths. One simple way to determine the glucose concentration using both ranges of wavelengths is to calculate a value for glucose concentration based on the correlations between transmitted signal and glucose concentration, and then average the two values. This will reduce the uncertainty margins by using two independent ranges of wavelengths. However, there is a significant amount of non-linearity in the correlation between the signals and the glucose concentration in the biological material. Commonly used regression-based models do not possess the capability to incorporate such large amount of non-linearity, consequently leading to inaccurate predictions. With data acquisition using radio frequencies and infrared wavelengths, the complexity of the generated data will skew the results using such simplified models. This can be addressed with models using artificial intelligence that have wider capability to fit the acquired data for accurate predictions and detect outliers if any. Post-processing using machine learning is found to be more accurate than only using the detector signals or determining the glucose concentration using the simplified regression models. Further, AI models can take into account influences of other parameters even when the relationship is not known exactly. For example, the temperature dependence of glucose absorption might not be known, but including a temperature reading into the model will improve accuracy even without pre-establishing what the dependence is.

Using different wavelengths bands of radiation (optical and RF) also allows the method/system to utilize their different penetration depths, hence sampling different parts of the tissue and providing more information. This is not possible with a single wavelength band (e.g., optical) that may only sample the surface few millimeters.

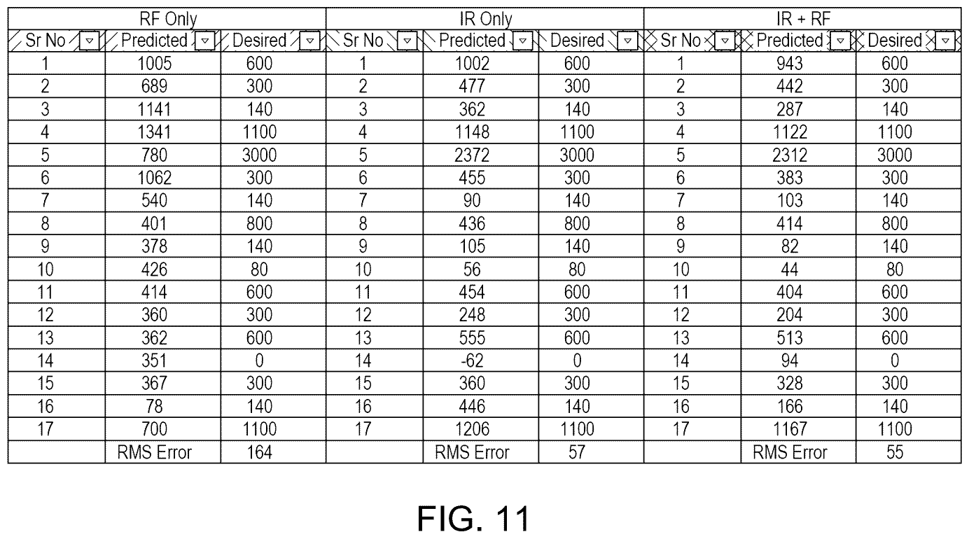

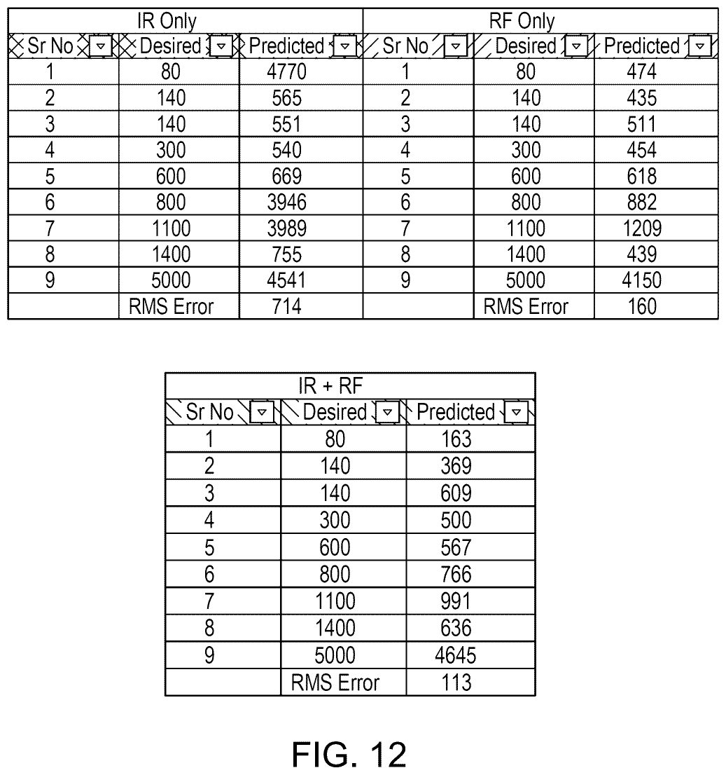

With reference to FIGS. 11 and 12, an artificial intelligence model can be trained with acquired data as explained above. FIG. 11 shows the results of a network trained using a training data set comprising 17 measurements and FIG. 12 shows the results of a network trained using a training data set comprising 9 measurements. The acquired data are divided into a training set and a test set. In an example, a machine learning model has 10 inputs and 1 output. The training data comprises, for each known value of glucose concentration in a biological material, an associated value of delta transmittance for each input (at a respective wavelength) of the optical radiation and a delta S21 s-parameter amplitude (delta with respect to S21 in water) for each input (at a respective wavelength) of the RF radiation. In this example, the inputs include 5 radio frequency signals at 35.99 GHz, 36.69 GHz, 37.39 GHz, 38.3 GHz and 38.72 GHz, 4 infrared wavelength signals at 962.21 nm, 1153.61 nm, 1370 nm and 1630 nm and 1 temperature signal detected for a sample. The output signal is glucose concentration and the network architecture of the machine learning module uses a technique called k-fold cross validation, in which the training set is divided into smaller sets. Each set is turn-wise reserved for testing, while the model is trained on the remaining data. This is repeated several times until all the data has been a part of the test set once. Using k-fold cross validation, it can be ensured that the model is not dependent on the data used for training and testing but is generalized for all the data available. The results for models trained using infrared signals only, RF signals only and both infrared and RF signals are shown in FIGS. 11 and 12. By using both signals from the infrared and RF radiation significantly improves the results as measured by the root-mean square error from the known values of glucose concentration from of the acquired data.

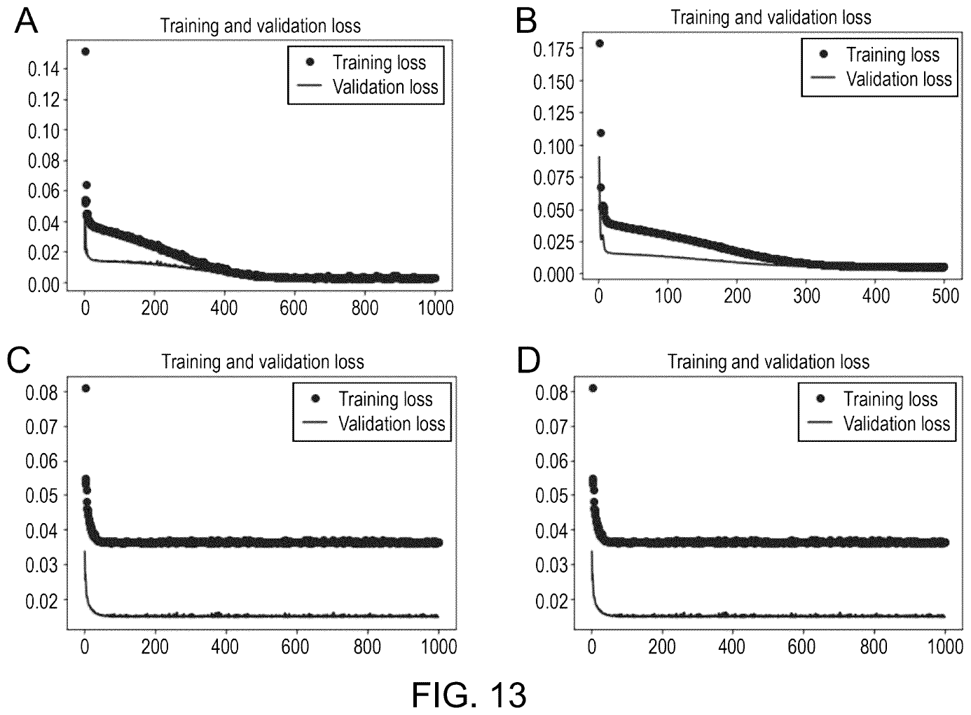

Although the models described above use 10 inputs in order to determine the glucose concentration, the same principles apply to models having fewer inputs. Having fewer inputs may reduce the accuracy of the model to an extent, but also simplifies the hardware for obtaining the signals, reducing the difficulty to engineer a lightweight and non-invasive sensing system and the corresponding cost savings. Accordingly, further models having fewer inputs provide attractive compromises by having fewer inputs but still providing the improved accuracy by having at least one RF radiation input and at least one optical radiation input. Example configurations of the models along with the training and validation loss is shown in the FIGS. 13A, 13B, 13C and 13D. FIG. 13A shows results of a model using 1 RF radiation input and 4 IR radiation inputs. FIG. 13B shows results of a model using 1 RF radiation input at 8.2 mm wavelength and 2 IR radiation inputs at 1370 nm and 1630 nm wavelengths. FIG. 13C shows results of a model using 1 RF radiation input at 8.2 mm wavelength and 1 IR radiation inputs at 1370 nm. FIG. 13D shows results of a model using 1 RF radiation input at 8.2 mm wavelength and 1 IR radiation inputs at 1630 nm wavelength.

With reference to FIGS. 13A, 13B, 13C and 13D, the model can be chosen based on the particular need of the application for the model, e.g., if the additional complexity is not an issue but increased accuracy is important a higher input model can be chosen. One good compromise for glucose concentration determination is the 2 IR, 1 RF model for which the results are shown in FIG. 13C, which requires only three inputs and therefore a relatively simple set up, but still provides good accuracy for determining glucose concentration.

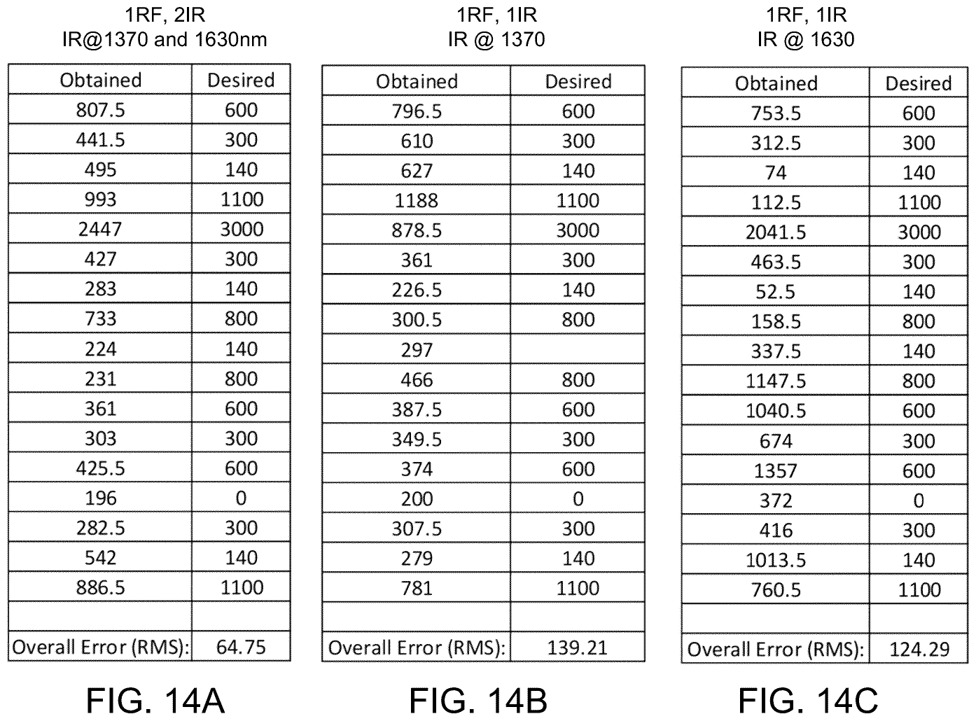

With reference to FIGS. 14A, 14B and 14C, the advantage of using a 2 IR, 1 RF input model reduces the overall error significantly compared to using only one of the two IR inputs with 1 RF input. FIG. 14A shows the results from a model using 1 RF radiation input at 8.2 mm wavelength, two IR inputs at 1370 and 1630 nm wavelength and temperature as an input. FIG. 14B shows the results from a model using 1 RF radiation input at 8.2 mm wavelength, one IR input 1370 nm wavelength, and temperature as an input. FIG. 14C shows the results from a model using 1 RF radiation input at 8.2 mm wavelength, one IR input at 1630 nm wavelength, and temperature as an input. Using both 1370 and 1630 nm wavelengths reduces the overall error by approximately a half.

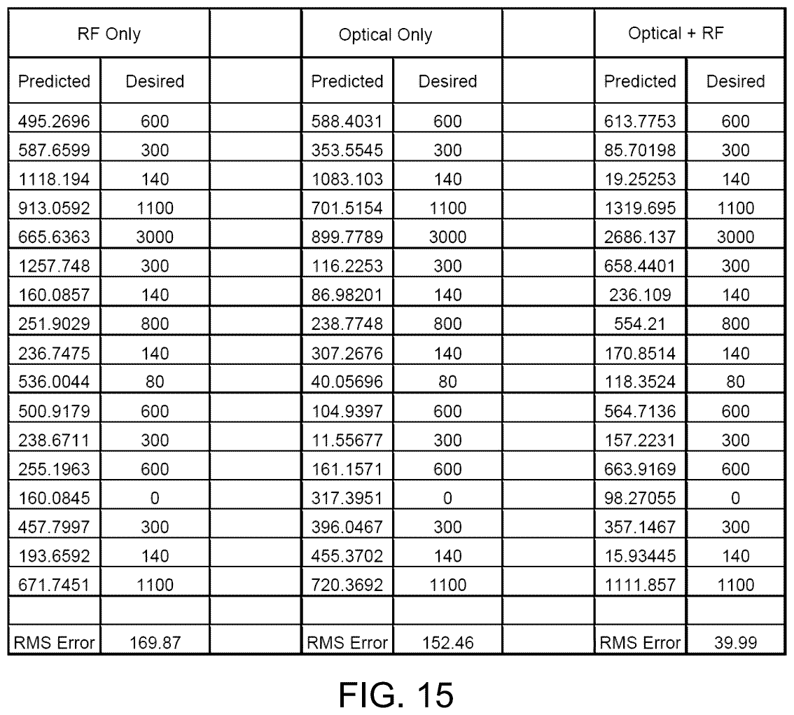

With reference to FIG. 15, the advantage of using a model with both optical and RF inputs reduces the overall error significantly compared to using only one of optical or RF inputs. In this example, the inputs to the model were: one RF radiation input at 8.2 mm only (for the table heading “RF only”); one optical radiation input at 1370 nm wavelength only (for “optical only”); and the two inputs in combination (for “optical+RF”). The RF radiation input can be in a range from 8.1 mm to 8.3 mm, and the optical radiation input can be in a range from 1360 nm to 1380 nm. Using both optical and RF wavelengths reduces the overall error by a factor of approximately 4.

Impedance Sensing

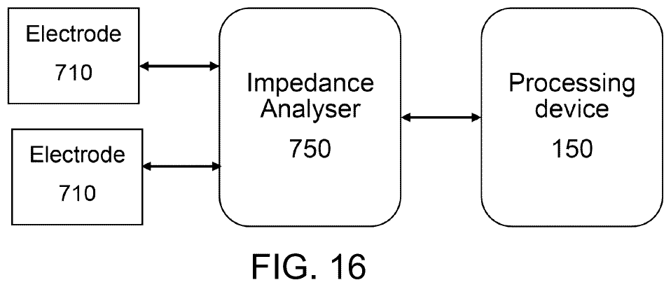

With reference to FIG. 16, an example of an auxiliary sensor is an impedance sensor. With reference to FIG. 16, the impedance sensor 700 comprises two electrodes 710 and an impedance analyzer 750. The impedance analyzer is configured to communicate an auxiliary signal which communicates the measured impedance to the processing device 150. For example, the auxiliary signal is communicated via an I2C (Inter-Integrated Circuit) serial communication bus or alternative wired or wireless communication protocol.

The electrodes 710 are each arranged in contact with the biological material (not shown) at a contact region. Accordingly, an electrical signal can pass to and from the biological material via the electrodes. The impedance analyzer is configured to provide an electrical signal to one or both of the electrodes to measure an electrical impedance of the biological material (i.e., a “bioimpedance”) at the contact region. For example, the electrical signal can be an alternating current signal at 50 kHz and 600 mV amplitude (peak to peak). One of the electrodes may be a reference electrode. The impedance can be measured by detecting the current supplied to and received from the biological material via the electrodes and the voltage across the electrodes and calculating the impedance from current and voltage. The phase of a received signal compared to the supplied electrical signal can also be used when measuring the impedance. The two or more electrodes 710 of the impedance sensor may be positioned adjacent to the optical sensor 125 and RF sensor 135 of the sensor system.

The impedance measured by the impedance analyzer can be understood analogously to resistance for direct current. As such, the impedance is lower when electrical current flow is less inhibited by its medium. For example, the impedance between electrodes decreases as the area of contact between the electrode and biological material increases (i.e., the contact region increases in size), or the contact pressure between the electrodes and biological material increases, or there are fewer/smaller airgaps between the electrodes and the biological material.

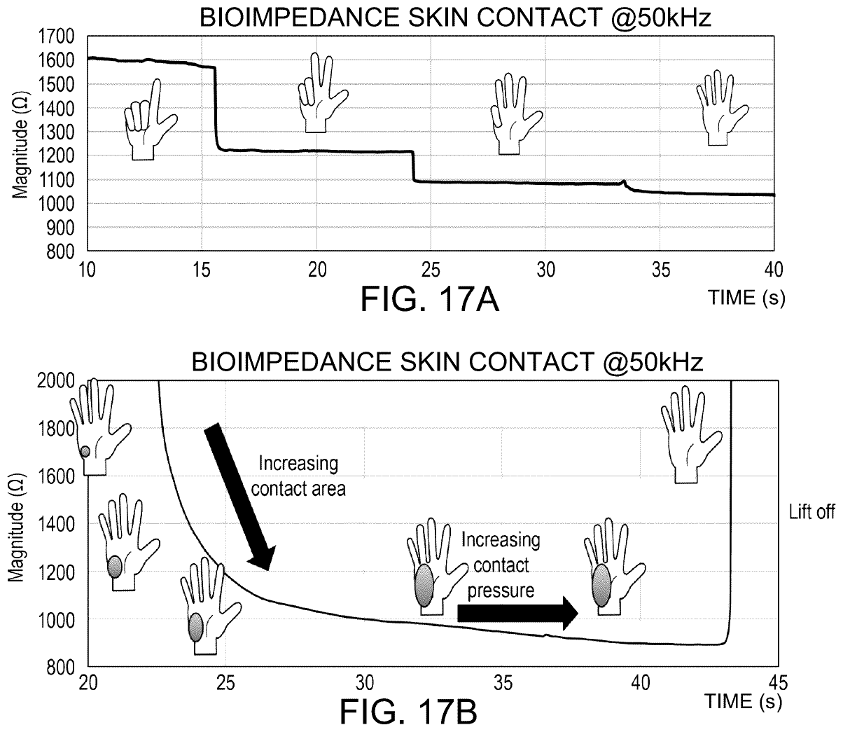

FIGS. 17A and 17B show two plots of bioimpedance for electrodes in contact with a hand under changing circumstances over time. FIG. 17A shows the impedance decrease as the electrodes contact one, two, three and then four fingers, respectively. The impedance changes from approximately 1600Ω (one finger, up to time marker 16 s), approximately 1210Ω (two fingers, 16 s to 24 s), approximately 1090Ω (three fingers, 24 s to 33 s) and 1040Ω (four fingers 33 s to 40 s). FIG. 17B shows the impedance decrease from above 2000Ω to approximately 1000Ω with increasing contact area (20 s to 30 s) between a palm and the electrodes, and further down to approximately 900Ω with increasing contact pressure (30 s to 43 s). When the contact between the electrodes and hand is removed, i.e., the hand is lifted off, the impedance immediately increases again as the there is no longer an effective conductor between the electrodes.

By using an impedance sensor as an auxiliary sensor, the sensor system and methods for measuring glucose concentration in a biological material can have improved accuracy. This is because penetration of the irradiation of the biological material with the optical radiation and the RF radiation is affected by the contact between the respective sensors and the biological material. For example, correct alignment of the sensors increases the quality of the received signals, whereas motion of the sensors may cause signal instabilities and air gaps between a sensor and the biological material can alter the signal, especially if the affects vary during a measurement or between measurements. By taking into account the measured impedance between electrodes and the biological material, which is dependent on the contact area, contact pressure, and any air gaps, these potential errors in the optical and RF readings can be identified and removed. For example, the impedance can be used as an additional input into a machine learning model as defined above, which would reduce the error in the glucose concentration determination. Alternatively, the impedance measurement can be a check for adequate contact before the optical and RF sensing begins.

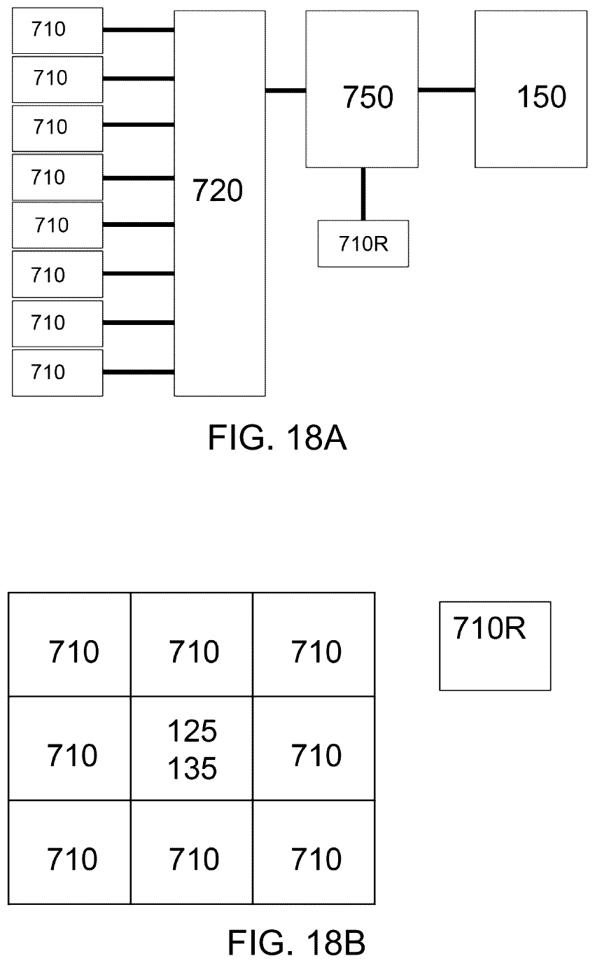

With reference to FIGS. 18A and 18B, the impedance sensor may comprise more than two electrodes. With reference to FIG. 18A, an impedance sensor comprises eight sample electrodes 710 and one reference electrode 710R. A switch 720 is configured to sample different pairs of the electrodes sequentially for the impedance analyzer 750 to measure impedance and communicate to the processing device 150. Each respective pair of electrodes within the set of electrodes will provide an impedance reading for a slightly different contact region between the two electrodes of each respective pair. Having more than two electrodes total will therefore provide values of impedance covering a larger contact area, or provide a more accurate value by averaging several impedance readings. With reference to FIG. 18B, in an example there are eight electrodes 710 arranged adjacent and surrounding the optical sensor 125 and RF sensor 135. A reference electrode 710R may be included in the array of electrodes 710, or positioned separately.

An impedance as described above can be used instead of or in addition to any of the auxiliary sensors described above with reference to sensor systems, sensor devices, and methods for measuring glucose concentration.

Computer-Readable Medium

The various methods described above may be implemented by a computer program product. The computer program product may include computer code arranged to instruct a computer to perform part of the functions of one or more of the various methods described above. The computer program and/or the code for performing such methods may be provided to an apparatus, such as a computer or processing device as described above, on a computer readable medium or computer program product. The computer readable medium may be transitory or non-transitory. The computer readable medium could be, for example, an electronic, magnetic, optical, electromagnetic, infrared, or semiconductor system, or a propagation medium for data transmission, for example for downloading the code over the Internet. Alternatively, the computer readable medium could take the form of a physical computer readable medium such as semiconductor or solid-state memory, magnetic tape, a removable computer diskette, a random-access memory (RAM), a read-only memory (ROM), a rigid magnetic disc, and an optical disk, such as a CD-ROM, CD-R/W or DVD.

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

1-35. (canceled)

36. A method for measuring glucose concentration in a biological material, the method comprising:
irradiating the biological material with optical radiation having a first wavelength between 400 nanometers and 25 micrometers;
detecting a first signal from the biological material at the first wavelength;
irradiating the biological material with radio-frequency (RF) radiation having a second wavelength between 1 millimeter and 30 centimeters;
detecting a second signal from the biological material at the second wavelength; and
determining a concentration of glucose in the biological material based on the first signal and the second signal.

37. The method of claim 36, wherein the first wavelength is between 780 and 2500 nm.

38. The method of claim 36, wherein the second wavelength is less than 2 centimeters.

39. The method of claim 36, wherein the irradiating the biological material with optical radiation further comprises:
irradiating the biological material at a third wavelength between 400 nanometers and 25 micrometers, wherein an interaction of glucose with the optical radiation at the third wavelength is independent of the concentration of glucose; and
detecting a third signal at an optical radiation detector, wherein the determining the concentration of glucose is further based on the third signal.

40. The method of claim 39, wherein the third wavelength is between 1400 and 1550 nm.

41. The method of claim 36, wherein the first signal is a reflection signal reflected off the biological material.

42. The method of claim 36, wherein the first signal is a transmission signal transmitted through the biological material.

43. The method of claim 1, further comprising:
modulating the optical radiation and the RF radiation, wherein the modulating is selected from the group consisting of: amplitude modulation, frequency modulation, and phase modulation.

44. The method of claim 43, further comprising:
creating a fast Fourier transform (FFT) of the second signal, wherein the determining the concentration of glucose includes using the FFT of the second signal.

45. The method of claim 36, wherein the biological material has a thickness, wherein the first wavelength is longer than the thickness of the biological material, and wherein the second wavelength is shorter than the thickness of the biological material.

46. The method of claim 36, wherein the determining the concentration of glucose involves inputting the first signal and the second signal into a machine learning model based on the first signal at approximately 1370 nm and approximately 1630 nm and the second signal at approximately 8.2 mm.

47. The method of claim 36, wherein the biological material is selected from the group consisting of: a web of skin between a finger and a thumb, an earlobe, an armpit, a wrist, a lip, and a foodstuff.

48. A sensor system for measuring glucose concentration in a biological material, the sensor comprising:
an optical radiation source that irradiates the biological material with optical radiation having a first wavelength between 400 nanometers and 25 micrometers;
an optical radiation detector that detects a first signal from the biological material at the first wavelength;
a radio-frequency (RF) radiation source that irradiates the biological material with RF radiation having a second wavelength between 1 millimeter and 30 centimeters;
an RF radiation detector that detects a second signal from the biological material at the second wavelength; and
a processing device configured to determine the concentration of glucose in the biological material based on the first signal and the second signal.

49. The sensor system of claim 48, further comprising:
a holder that secures the biological material in a fixed position with respect to the optical radiation source, the optical radiation detector, the RF radiation source, and the RF radiation detector.

50. The sensor system of claim 49, wherein the holder is configured to secure the optical radiation source and the optical radiation detector on opposite sides of the biological material, and to secure the RF radiation source and the RF radiation detector on opposite sides of the biological material.

51. The sensor system of claim 49, wherein the first signal is a reflection signal, and wherein the holder is configured to secure the optical radiation source and the optical radiation detector on the same side of the biological material.

52. The sensor system of claim 48, wherein the holder comprises a wearable strap for attaching to the biological material.

53. The sensor system of claim 48, further comprising:
an optical filter disposed between the biological material and the optical radiation detector, wherein the optical filter transmits the first wavelength and filters out other wavelengths.

54. The sensor system of claim 48, wherein the RF radiation source and the RF radiation detector are a single antenna.

55. The sensor system of claim 48, further comprising:
a communication interface coupled between the RF radiation detector and the processing device, wherein the communication interface comprises a wireless connection.
